(19) United States
(12) Patent Application Publication
(10) Pub. No.: US 2008/0160509 A1
(43) Pub. Date: Jul. 3, 2008

(54) ASSAYS FOR PREDICTING AND ASSESSING PREDISPOSITION TO MALE PATTERN BALDNESS

(75) Inventors: Robert E. Akridge, Seattle, WA (US); Kenneth A. Pilcher, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: PACIFIC BIOSCIENCE LABORATORIES, INC., Bellevue, WA (US)

(21) Appl. No.: 12/048,139

(22) Filed: Mar. 13, 2008

Related U.S. Application Data
(63) Continuation-in-part of application No. 11/531,488, filed on Sep. 13, 2006.

(51) Int. Cl. C12Q 1/70

(52) U.S. Cl. 435/5

(57) ABSTRACT

A method of predicting the likelihood that a subject will develop alopecia, a method for identifying an individual as a candidate for treatment of androgenetic alopecia, a method for identifying a male subject infected with HSV as a candidate for treatment of androgenetic alopecia, a method of managing a treatment of androgenetic alopecia in a subject, and a kit for predicting or monitoring androgenetic alopecia in a subject are described.

### CROSS-REFERENCE TO RELATED APPLICATION

This application is a continuation-in-part of prior application Ser. No. 11/531,488, filed Sep. 13, 2006, priority from the filing date of which is hereby claimed under 35 U.S.C. § 120.

### BACKGROUND

Hair loss, though not life threatening, has profoundly negative social and psychological impacts. It is estimated that in the United States, about 35 million men younger than age 50 have discernable hair loss. The market demand for hair loss prevention and regrowth products is a multi-billion dollar segment. Although existing hair loss treatments are somewhat effective, there are no products that can entirely activate hair growth or prevent male pattern baldness.

Two drugs are FDA approved for the treatment of hair loss. The drug minoxidil is thought to work by increasing blood vessel formation to the hair follicle. Minoxidil is most effective on the crown region of the head and less effective on the frontal region. Treatment with minoxidil may result in the growth of some hair, but is generally more effective for retaining existing hair. The drug finasteride is prescribed as a lower dose of a drug that shrinks the prostate. Finasteride has been shown to retain existing hair, and may contribute to some growth and/or thickening of the hair. Unfortunately, these products do not work for all users, and the level of improvement is minimal for many individuals. Moreover, any improvement is often lost once treatment is discontinued.

It is widely accepted that hair loss has a genetic component. The so-called “baldness gene” triggers overproduction of 5α-reductase, the enzyme that converts testosterone to 5α-dihydrotestosterone (DHT). The enzyme 5α-reductase exists in two isoforms (type 1 and type 2). Of these two isoforms, type 1 is found in scalp skin, whereas type 2 is the predominant form in prostate. Testosterone is converted to DHT by 5α-reductase in several organs including the prostate, hair follicles, skin, liver, and sebaceous glands. DHT is known to be a potent androgen in androgenetic alopecia, as well as in benign prostatic hyperplasia, or enlargement of the prostate. It has been shown that approximately 90% of bald and balding men have elevated levels of DHT in their scalp. However, more than 70% of men with hair also have elevated levels of DHT. This finding suggests that there are additional factors that contribute to androgenetic alopecia and male pattern baldness.

### SUMMARY

The invention relates to a method of predicting the likelihood that a subject will develop alopecia. The method comprises:

(a) determining a presence of a nerve dwelling virus in a subject;

(b) determining a level of at least one of 5α-dihydrotestosterone, 5α-reductase, or testosterone in the subject;

(c) comparing the level of 5α-dihydrotestosterone, 5α-reductase, or testosterone determined from step (b) to a predetermined value; and

(d) predicting alopecia or a predisposition to alopecia in the subject, wherein the level of 5α-dihydrotestosterone, 5α-reductase, or testosterone in comparison to the predetermined value and the presence of the nerve dwelling virus is indicative of an increased likelihood of the subject developing alopecia.

In one embodiment, the nerve dwelling virus is herpes simplex virus.

In one embodiment, the presence of the nerve dwelling virus in the subject is determined by an assay selected from an immunological assay, a polymerase chain reaction assay, or an RNA-DNA microarray assay.

In one embodiment, the level of 5α-dihydrotestosterone, 5α-reductase, or testosterone in the subject is determined by an assay selected from an immunological assay, a polymerase chain reaction assay, or an RNA-DNA microarray assay.

In one aspect, the invention provides a method for identifying an individual as a candidate for treatment of androgenetic alopecia. The method comprises:

(a) obtaining a positive test result for a herpes simplex virus in an individual;

(b) determining the level of at least one of 5α-dihydrotestosterone or 5α-reductase;

(c) comparing the level of 5α-dihydrotestosterone or 5α-reductase to a predetermined value; and

(d) characterizing the individual as a candidate for treatment of androgenetic alopecia based upon a positive test result in step (a) and an elevated level of 5α-dihydrotestosterone or 5α-reductase in comparison to the predetermined value.

In one embodiment, the herpes simplex virus is HSV-1.

In one aspect, the invention provides a method for identifying a male subject infected with herpes simplex virus as a candidate for treatment of androgenetic alopecia. The method comprises:

(a) measuring a level of at least one of 5α-dihydrotestosterone or 5α-reductase in a male subject infected with herpes simplex virus; and

(b) comparing the level measured in step (a) to a reference, wherein a level above the reference indicates that the male subject is a candidate for treatment of androgenetic alopecia.

In one aspect, the invention provides a method of managing a treatment of androgenetic alopecia in a subject. The method comprises:

(a) measuring the presence of active herpes simplex virus replication in a subject;

(b) measuring a level of at least one of 5α-dihydrotestosterone or 5α-reductase in the subject;

(c) comparing the level from step (b) to a predetermined value; and

(d) adjusting the treatment of the subject to suppress replication of the herpes simplex virus or to reduce the level of at least one of 5α-dihydrotestosterone or 5α-reductase.

In one embodiment, the herpes simplex virus is HSV-1.

In one aspect, the invention provides a kit for predicting or monitoring androgenetic alopecia in a subject. The kit includes the following components:

(a) at least one reagent for detecting herpes simplex virus in a biological sample;

(b) at least one reagent for detecting or measuring the level of 5α-dihydrotestosterone or 5α-reductase in a biological sample;

(c) a reference standard for the assay of step (b);

(d) a set of instructions describing how to assay the biological samples; and

(e) a chart for determining the likelihood of the subject developing androgenetic alopecia based upon the test results obtained from the biological samples.

In one embodiment of the kit, the herpes simplex virus is HSV-1.

In one embodiment of the kit, the kit comprises at least one collection device.

### DETAILED DESCRIPTION

The present invention relates to a method of predicting a likelihood that a subject will develop alopecia, a method for identifying an individual as a candidate for treatment of androgenetic alopecia, a method for identifying a male subject infected with herpes simplex virus as a candidate for treatment of androgenetic alopecia, a method of managing a treatment of androgenetic alopecia in a subject, and a kit for detecting or monitoring androgenetic alopecia in a subject.

The methods and kit described herein are useful for determining if a subject is predisposed to hair loss, and if hair loss treatment is sufficient to alter the progression of hair loss.

Alopecia is the loss of hair from the head or body. Androgenetic alopecia (AGA) is a common type of hair loss in both men and women. In men, this condition is commonly referred to as male pattern baldness (MPB). The terms AGA and MPB are used interchangeably herein. In subjects with MPB, hair is lost in a well-defined pattern beginning above both temples. Over time, the hairline recedes to form a characteristic “M” shape. Hair also thins at the crown of the head, often progressing to partial or complete baldness. In order to classify the severity of hair loss, scales categorizing various stages of hair loss have been developed. One example is the Hamilton-Norwood scale, which ranges from stages I to VII, with stage I being the least severe and stage VII being the most severe form of hair loss. The Hamilton-Norwood scale is described in D. Norwood, Male Pattern Baldness: Classification and Incidence, South. Med. J. 68(11):1359-65 (1975).

In the past, baldness has been considered to be strictly genetically inherited. However, it has been observed that hair loss and hair regrowth (or the lack thereof) are tightly linked to a chronic viral infection, viral replication, and the associated immune response. The cause of hair loss is believed to be related to an initial viral infection which comes to dwell in the nerves of the scalp. This chronic viral infection is caused by a nerve-dwelling virus, for example, Herpes Simplex Virus-1 (HSV-1), an HSV clade or mutant form, or a similar nerve dwelling virus. The initial infection is followed by activation of latent viral replication, which is followed in turn by an immune response to the viral infection. Viral replication and the subsequent immune response are heightened in the scalp of males after puberty (due to the onset of testosterone biosynthesis) if they are genetically predisposed to produce elevated levels of 5α-reductase. The genetically determined, elevated level of 5α-reductase in the scalp converts testosterone to DHT, resulting in elevated levels of DHT. Elevated levels of DHT result in increased viral replication at the site of biosynthesis. The heightened level of virus due to DHT results in an amplified antiviral immune response that is believed to eventually weaken the hairs. Each immunological episode wherein the virus goes from latent stage to active stage and is forced back into the nerve by the amplified immune response is thought to result in hair fallout and loss. Thus, in virally infected post-pubescent males, the conversion of testosterone to DHT increases viral replication when viral latency is broken and is thought to produce continued immunological action against hair follicles. The connection between hair loss, hair regrowth, and chronic viral infection is described in U.S. application Ser. No. 11/531,488, the disclosure of which is incorporated herein by reference.

Baldness and the degree of baldness are further indicated as being linked to viral acquisition prior to puberty. Once puberty occurs, the elevated level of testosterone in males is normally converted to DHT. Men who are genetically predisposed to having high levels of 5α-reductase, the enzyme that converts testosterone to DHT, are believed to be more likely to become bald if they are infected with a virus that dwells in the nerves of the scalp. In these men, baldness may be limited to the areas serviced by nerves and nerve branches where the latent virus resides. Thus, baldness is believed to be caused by a chronic viral infection, its future replication at some point in time, and the associated immune response of the body to the virus. In accordance with the present invention, methods for detecting the presence and concentrations of the key balding agents (HSV, 5α-reductase, and DHT) are used to predict the likelihood of developing hair loss, a suitable method of treatment, and the likely effectiveness of treatment to suppress the key balding agents.

Androgenetic alopecia affects men and women. It is possible that hair loss in older women is due to a viral infection, as observed in males, but that there is no effect on hair loss until a hormonal shift in later years results in increased testosterone. In females who are genetically predisposed for higher levels of testosterone, and who also have a nerve dwelling virus, the hormonal shift may contribute to a more pronounced immune response in the scalp and loss of hair. Therefore, methods described herein can also be used to predict, assess, and monitor hair loss in females.

Prediction of Androgenetic Alopecia. In one aspect, the invention provides a method of predicting the likelihood that a subject will develop alopecia. The method comprises:

(a) determining a presence of a nerve dwelling virus in a subject;

(b) determining a level of at least one of 5α-dihydrotestosterone, 5α-reductase, or testosterone in the subject;

(c) comparing the level of 5α-dihydrotestosterone, 5α-reductase, or testosterone determined from step (b) to a predetermined value; and

(d) predicting alopecia or a predisposition to alopecia in the subject, wherein the level of 5α-dihydrotestosterone, 5α-reductase, or testosterone in comparison to the predetermined value and the presence of the nerve dwelling virus is indicative of an increased likelihood of the subject developing alopecia.

In one embodiment, predicting the likelihood that a subject will develop androgenetic alopecia comprises detecting the presence of a nerve dwelling virus (e.g. HSV-1) and its replication, and detecting elevated levels of DHT. Elevated DHT levels exist in individuals that have androgenetic alopecia, however these high levels also exist in males without AGA (in this case, the individuals are likely not infected with HSV-1). It is believed that males that have HSV-1 but are not bald have normal levels of DHT. It is thought that only when both of these factors (virus and elevated DHT levels) are present, that AGA occurs.

In order to predict the likelihood of developing AGA, two serum specimens would be drawn from the subject. The first serum specimen would be processed using a HSV-1 ELISA to check for HSV-1 antibodies. HSV ELISAs are known in the art, and are described in more detail below. As a first control group, a serum pool from AGA subjects known to have high viral titers would be run in parallel with the subject's serum specimen. Another control group would be pooled serum from non-bald subjects that are HSV-1 negative.

The second serum specimen from the subject would be processed to check for the concentration of DHT using ELISA. As a first control group, a serum pool from AGA subjects known to have high levels of DHT would be run in parallel with the subject's specimen. Because DHT concentration varies with age, age-appropriate pooled sera would be used. Another control group would be pooled serum from non-bald subjects without elevated levels of DHT. In this way, the predetermined or reference value of DHT for use in the methods of the invention could be obtained. If the patient's DHT levels fall within the predetermined or reference value of the DHT levels of the pooled serum control group of AGA individuals, and the patient is also positive for HSV-1 antibodies, then the patient is at high risk of developing AGA.

In one embodiment, the predetermined value of 5α-reductase or testosterone could be obtained using a methodology and control groups similar to the methodology used for obtaining the predetermined value of DHT. In the methods, other specimens (e.g., scalp biopsies, topical swabs) could be used instead of serum specimens. Similar pooled specimens would be needed for the controls. If the patient's levels of 5α-reductase or testosterone fall within the predetermined or reference value of the levels of 5α-reductase or testosterone of the pooled serum control group of AGA individuals, and the patient is also positive for HSV-1 antibodies, then the patient is at high risk of developing AGA.

A threshold level of DHT needed to slow the progression of AGA has been established (K. D. Kaufman and R. P. Dawber, Finasteride, a Type 2 5α-Reductase Inhibitor, In the Treatment of Men with Androgenetic Alopecia, Expert Opin. Invest. Drugs, April; 8(4):403-15 (1999); see also L. Drake et. al., The Effects of Finasteride on Scalp Skin and Serum Androgen Levels in Men with Androgenetic Alopecia, J. Am. Acad. Derm. 41(4):550-4 (1999)). It is important to maintain patients at the DHT threshold level and possibly even lower if the viral titer is seen to remain elevated after treatment.

In one embodiment, the predetermined or reference value for use in the methods of the invention is an average value of the level of DHT measured in a pool of samples obtained from HSV positive bald subjects, or an average value of DHT measured in a pool of samples obtained from HSV positive non-bald subjects. In such an embodiment, the DHT level of an HSV positive test subject may be compared to the DHT reference value obtained from the HSV positive bald subjects, and/or to the DHT reference value obtained from the HSV positive non-bald subjects. If the DHT level of the HSV positive test subject is more similar to the reference value obtained from the HSV positive bald subjects, the test subject is more likely to develop AGA. Conversely, if the DHT level of the HSV positive test subject is more similar to the reference value obtained from the HSV positive non-bald subjects, the test subject is less likely to develop AGA.

Herpes Simplex Virus. In one embodiment, the nerve dwelling virus is HSV. There are nine known herpes viruses. All nine types are characterized by having a double-stranded DNA genome. Two of the best known herpes viruses are HSV-1 and HSV-2. HSV-1 is commonly known as the “kissing virus” because it is transmitted by oral contact. Transmission usually occurs in childhood when an adult with an oral lesion kisses a child on the lips. After infection the virus remains latent in the nerves, and only replicates when latency is broken. When activated, the virus leaves the nerve endings to infect and replicate in adjacent cells. The immune response to viral activation and replication can include symptoms such as oral lesions (cold sores), fever, and local tenderness. Although approximately 80% of the general population in the U.S. is infected with HSV-1, the virus remains dormant and does not cause oral lesions in two-thirds of those infected. It is in only about one-third of the infected population that the residual virus particles become active enough to cause recurrent cold sore or fever blister outbreaks.

The other well-known HSV virus is HSV-2. HSV-2 is sexually transmitted and causes genital herpes. Lately, HSV-1 has been shown to also cause genital herpes, and the distinction between the diseases caused by HSV-1 and HSV-2 has become less clear.

HSV is sequestered in specific nerves of the nervous system. HSV-1 typically resides in the trigeminal nerve. Restriction of a virus to a particular nerve and restriction of the pathology of a disease to a specific area of the body is not unknown. For example, varicella-zoster virus, which is another type of herpes virus, causes “shingles” adjacent to the infected nerve of the back or on parts of the face. Likewise, HSV-1 resides in the trigeminal nerve and results in lesions where the virus exits to branches of the trigeminal nerve. The virus in the third branch of the nerve, which covers the scalp, does not result in blisters but is believed to manifest itself in the form of hair loss.

Detection of HSV-1 and HSV-1 Antibodies. In one embodiment, the presence of a nerve dwelling virus in the patient is determined by an assay selected from an immunological assay, a polymerase chain reaction assay, or an RNA-DNA microassay. For example, there are multiple versions of immunological assays for detection of HSV-1 consisting of different competitive binding scenarios. Usually competition occurs between an antigen (e.g., HSV-1 protein) which is pre-adhered to a specific substrate (e.g., nitrocellulose membrane) and a specific primary antibody (e.g., anti-HSV-1 IgG antibody) from a specimen (e.g., serum). After a specific incubation period the substrate is washed to remove all unbound material, and a secondary labeled antibody is added that is specific for the primary antibody (e.g., anti-IgG-HRP). After another period of incubation and washing the enzyme substrate is added. The enzyme reaction is then stopped and the resulting colored solution absorbance is measured. A set of known standards concentrations is used determine the concentration within the specimen. Variations on this theme exists with the substrate consisting of polystyrene or pre-coated dipsticks, beads, filter paper or any substrate known to bind proteins.

The order of what is being captured (and thus what is revealed) is variable. For example, instead of binding the antigen to the substrate and detecting the presence of an antibody (e.g., anti-HSV-1), the specimen can be allowed to bind to the substrate. The desired antigen (e.g., HSV-1) is then bound and subsequently detected by HSV-1 specific antibodies that are labeled, thereby revealing the presence of the virus itself. Because the virus titer is often at the low end of the assay detection limits, other labels such as fluorescent probes and radioactive isotopes are used in place of enzymes to boost the signal. Assays to detect viral levels are known in the art, and examples of these types of assays are described in H. Dordevic, Serological Response to Herpes Simplex Virus Type 1 and 2 Infection Among Women of Reproductive Age, Med. Pregl. 59(11-12):591-97 (2006); see also A. I. Fusun et al. Distribution of HSV-1 IgG Antibodies by Two Methods Comparing in Turkish Atopic Children, New Microbiol. 30(2):109-12 (2007).

Although enzyme link immunosorbent assays (ELISA) are a well-known methodology for detection of specific targets (e.g., DHT), and infectious agents (e.g., HSV), one of skill in the art would know that other scientific methodologies could also be used. These other methods include, but are not be limited to, polymerase chain reaction (PCR) assays, electrophoresis methods, Western blots, dot-blots, radioactive labeling assays, immunofluorescent assays, and RNA-DNA microarray assays. For example, microarray assays allow a large number of genes to be scanned for activation. Viruses cause a specific pattern of activated genes. By tracking the gene pattern, and not the virus, it would be possible to determine if the viral level present is sufficient to cause gene activation, which could then lead to hair loss. Even though kit and rapid bench top methods of analysis would be the simplest method for evaluating MBP, other more advanced techniques could also be used, for example, gas chromatography-mass spectrometry.

5α-Dihydrotestosterone. DHT is a steroid similar to testosterone and androstenedione, which belong to a class called androgens. DHT is a C19 steroid and possesses androgenic activity. The bulk of androgen production takes place mainly in the Leydig cells of the testes. Androgens circulate in the blood bound to proteins, especially sex hormone binding globulin (SHBG) and albumin. A trace amount of these steroids circulate in the unbound form in the blood and are referred to as the free fractions. DHT has at least three times the binding affinity for SHBG than testosterone. In males about 70% of DHT is derived from peripheral conversion of testosterone, while in females most of the DHT is derived from androstenedione.

The major organ to neutralize androgens is the liver. In the liver the steroid hormones undergo structural modifications that are generally regarded as prerequisites for their biological inactivation. Metabolites are formed and then returned to circulation before renal excretion. Therefore, elimination of steroids from the body is done through the urine.

The DHT level in young people is much higher than the level found in normal older people, hence androgen production increases at puberty which gives rise to masculinizing characteristics. It has been demonstrated that the human testes produce DHT, which appears to originate in the seminiferous tubules. When there is tubular damage, the production of DHT is impaired, which causes a decrease in the levels of plasma DHT (patients with germinal cell aplasia and azoospermia). For example, there is a very low level of plasma DHT in patients with anorchia. It has been reported that in some prostate cancer (especially in stage D) the determination of DHT could be useful in predicting the response to anti-androgen therapy.

Detection of DHT and 5α-reductase. In one embodiment, the level of 5α-dihydrotestosterone or 5α-reductase in the patient is determined by an assay selected from an immunological assay, a polymerase chain reaction assay, or an RNA-DNA assay. There are several 5α-dihydrotestrone ELISAs used for DHT detection. A representative example is the ELISA available from Immunobiological Laboratories of Minneapolis, Minn. Similar antibodies (e.g., goat anti-SRD5A1) for 5α-reductase exist and are recommended for ELISA.

Assays for the detection of DHT and 5α-reductase are known in the art. Examples of these types of assays are described in A. L. Dallob et al., The Effect of Finasteride, a 5α-Reductase Inhibitor, on Scalp Skin Testosterone and Dihydrotestosterone Concentrations in Patients with Male Pattern Baldness, J. Clin. Endo. Metab. 79(3):703-06 (1994); H. Vierhapper et al., Production Rates of Dihydrotestosterone in Healthy Men and Women and in Men with Male Pattern Baldness: Determination by Stable Isotope/Dilution and Mass Spectrometry, J. Clin. Endo. Metab. 86(12):5762-764 (2001); and H. Licea-Perez et al., Development of a Highly Sensitive and Selective HPLC/MS/MS Method for the Simultaneous Determination of Testosterone and 5α-Dihydrotestosterone in Human Serum to Support Testosterone Replacement Therapy for Hypogonadism, Steroids [in press; doi:10.1016/j.steroids.2008.01.018].

Most enzyme-based assays and molecular analysis (e.g., PCR) use blood, serum, urine, saliva, or biopsies (e.g., scalp) for the detection of the desired antigen. In the case of diagnosis and monitoring the levels of virus (or antibody) and DHT/5-α reductase, these specimens would also be used. However, collection of the surface oils of the scalp and the tears could also be used. In a recent study it was determined that HSV-1 serum positive participants secreted virus in their tears, and intermittently in saliva, with most of the participants being asymptomatic for HSV-1.

Treatment of Hair Loss. Treatment as used herein means reduction or prevention of hair loss and/or promotion of new hair growth. In one aspect, the invention provides a method of identifying an individual as a candidate for treatment of androgenetic alopecia. The method comprises:

(a) obtaining a positive test result for a herpes simplex virus in an individual;

(b) determining the level of at least one of 5α-dihydrotestosterone or 5α-reductase;

(c) comparing the level of 5α-dihydrotestosterone or 5α-reductase to a predetermined value; and

(d) characterizing the individual as a candidate for treatment of androgenetic alopecia based upon a positive test result in step (a) and an elevated level of 5α-dihydrotestosterone or 5α-reductase in comparison to the predetermined value.

At times it will be known whether an individual is positive for HSV. In this case, it is possible to identify a male subject infected with herpes simplex virus as a candidate for treatment of AGA by measuring the level of at least one of 5α-dihydrotestosterone or 5α-reductase, and comparing the level of 5α-dihydrotestosterone or 5α-reductase to a reference. A level above the reference indicates that the male subject is a candidate for treatment of androgenetic alopecia.

In situations where individuals are identified as candidates for treatment of AGA, it is desired that the level of virus and DHT are kept under control (below a specific threshold). Methods for treating MPB are described in U.S. application Ser. No. 11/531,488, and generally include topical application of an antiviral agent to the scalp to suppress viral replication or viral activation present on the nerves in the region of the scalp which lead to hair loss. The antiviral agents that may be used for treatment include those that are commonly used for the treatment of herpes.

Although herpes cannot be cured, it can be treated. Treatment can speed up healing time of oral lesions, reduce pain, and delay or prevent additional flare-ups. Typically, treatment for herpes sores is used only during a flare-up. This type of therapy is called episodic therapy. In people with compromised immune systems, flare-ups can be frequent and may require long-term therapy to prevent recurrences. This type of therapy is called suppressive therapy.

There are four main drugs used for the treatment of herpes. The first of these drugs is acyclovir. Acyclovir has been studied and used for many years as a treatment for oral and genital herpes. It has been studied specifically in people with HIV and herpes and has been shown to be safe and effective. Acyclovir is available in a topical cream, pills, and an intravenous formulation. Most experts agree that the cream is not very effective and that pills are best for mild to moderate flare-ups or long-term suppressive therapy. Intravenous acyclovir is used to treat serious flare-ups or outbreaks that effect internal organs (especially HSV infection of the central nervous system). The oral dose used to treat flare-ups is 400 mg taken either three or four times a day, usually for seven to ten days. The dose can be doubled if the herpes sores fail to respond. Taking 400 mg of the drug three times daily or 800 mg of the drug twice a day for a prolonged period of time can help prevent flare-ups from recurring. However, this course of treatment is usually recommended only for patients who have a history of frequent recurrences.

Valacyclovir is a pro-drug of acyclovir and has been approved specifically for the treatment of herpes in HIV positive individuals. Unlike acyclovir, valacyclovir needs to be broken down by the body before its active ingredient—acyclovir—can begin controlling the disease. This allows for higher amounts of acyclovir to remain in the body, thus requiring a lower dose of the drug to be taken by mouth. For mild to moderate herpes flare-ups, valacyclovir only needs to be taken once a day by mouth (1000 mg daily). For episodic therapy, valacyclovir is taken for seven to ten days. However, the drug can be taken every day for a prolonged period of time using half the dose needed to treat flare-ups (500 mg every day). Like acyclovir, valacyclovir rarely causes side effects.

Famciclovir is the pill form of a topical cream called penciclovir. Treatment with famciclovir usually requires 500 mg taken by mouth for seven to ten days. A dose of 250 mg every day, taken for a prolonged period of time, is considered to be a safe and effective preventative therapy for recurrent herpes flare-ups.

Trifluridine drops are used to treat HSV infection of the eye(s). One drop is placed in the infected eye every two hours for up to 21 days. It cannot be used to treat or prevent HSV disease in other parts of the body.

In some cases, herpes flare-ups do not respond to acyclovir, valacyclovir, or famciclovir, probably due to the emergence of drug-resistant forms of HSV-1 and HSV-2. HIV positive patients with suppressed immune systems—usually having a T-cell count of less than 100—who have been receiving long-term acyclovir for the treatment and prevention of recurrent herpes flare-ups have been known to develop drug-resistant herpes. Because acyclovir is similar to both valacyclovir and famciclovir, simply switching to these two drugs is not usually effective.

At the present time, foscarnet is the most common treatment for acyclovir-resistant herpes. The drug must be administered via an intravenous line, usually three times a day, often in a hospital or under the close supervision of an in-home nurse.

In patients who are candidates for treatment of AGA, DHT levels should also be controlled. Currently the only approved DHT suppressor is finasteride. A 1 mg dose daily of finasteride is recommended to suppress DHT to safe levels with minimal side effects. This would be the first treatment given, followed by finasteride dose changes based on viral levels. Because DHT is thought to upregulate viral replication, a low viral titer after treatment with finasteride would indicate that the level is adequate.

As described herein, one aspect of hair loss prevention is the control of viral levels and outbreaks, which in turn suppresses the immune response and reduces the immunological action against hair follicles. Another approach to hair loss prevention is the direct suppression of the immune response, for example, the response that occurs in the scalp. The methods of immune system suppression are known to those of skill in the art and include, without limitation, suppression of the key inflammatory pathways such as the COX pathway.

At times it may be useful to manage the treatment of an subject with AGA. The method of managing treatment of androgenetic alopecia in a subject comprises:

(a) measuring the presence of active herpes simplex virus replication in a subject;

(b) measuring a level of at least one of 5α-dihydrotestosterone or 5α-reductase in the subject;

(c) comparing the level from step (b) to a predetermined value; and

(d) adjusting the treatment of the subject to suppress replication of the herpes simplex virus or to reduce the level of at least one of 5α-dihydrotestosterone or 5α-reductase.

In one embodiment, the following regimen could be used to maintain patients that have elevated levels of DHT and that are positive for HSV-1 below critical thresholds to prevent the progression of AGA. First, baseline levels of DHT and viral antibodies or virus would be determined as described above. Several samples would be taken over a period of several weeks. Next, finasteride and oral and topical antiviral agents would be prescribed. After two weeks, specimens (serum, topical swab or scalp biopsies) would again be collected to check for DHT levels compared to baseline and HSV-1 virus antibodies compared to baseline. Essential controls for both assays would consist of pooled serum from males (of similar age as the patient) that have MPB; pooled serum from males (of similar age) without MBP; pooled serum from males with HSV-1 (not on antivirals) and normal levels of DHT; pooled serum from males that are HSV-1 negative and have high levels of DHT; and eventually, pooled serum from males that have had positive results from treatment. Depending on the results of the assays, the prescribed doses of finasteride and/or antiviral agents would be adjusted to maintain the patient's levels of DHT and HSV below critical thresholds.

Adjusting the treatment of the subject could include adjusting the amount of antiviral agent alone or in combination with one or more additional therapeutically active agents (e.g., COX inhibitors) which in combination directly suppress both viral replication and the local immune response in the scalp.

In another aspect, the invention provides a kit for predicting or monitoring androgenetic alopecia in a subject. The kit comprises:

(a) at least one reagent for detecting herpes simplex virus in a biological sample;

(b) at least one reagent for detecting or measuring the level of 5α-dihydrotestosterone or 5α-reductase in a biological sample;

(c) a reference standard for the assay of step (b);

(d) a set of instructions describing how to assay the biological samples; and

(e) a chart for determining the of the likelihood of the subject developing androgenetic alopecia based upon the test results obtained from the biological samples.

The kit for predicting or monitoring AGA includes a chart. The chart is based upon the data shown in Table 1, below. As discussed herein, it is the combination of a virus on specific nerves to the scalp and a genetic predisposition to convert more testosterone to DHT that accounts for MPB. The degree of baldness is believed to be linked to viral acquisition prior to puberty, because hair loss will begin with elevated testosterone levels at puberty, with conversion to DHT. Referring to Table 1, below, a male who is infected with HSV-1 before puberty, who is also genetically predisposed to a high concentration of DHT, is believed to become bald (++). As further indicated in Table 1, if exposure to the virus occurs after puberty, it is believed that a male will lose hair at a different rate or in a different pattern than otherwise (+/−). Unless both factors are present, it is believed that a male will not lose hair in male pattern baldness. Females do not produce significant levels of DHT during the phase of their lifetime when AGA typically occurs, and therefore are not prone to the typical hair loss seen in MPB (−).

TABLE 1
Factors in Development of MPB

| | HSV-1 Before | Elevated | HSV-1 After | Will Become |
|---|---|---|---|---|
| SEX | Puberty | DHT Level | Puberty | Bald |
| Female | + | N/A | | − |
| Female | − | N/A | + | − |
| Male | + | + | | ++ |
| Male | + | − | | − |
| Male | − | + | | − |
| Male | − | + | + | +/− |
| Male | − | − | | − |

In one embodiment, the kit includes a collection device. The collection device has an embedded reagent so that at least one of the tests can be performed by the subject.

The following examples are provided for the purpose of illustrating, not limiting, the invention.

### EXAMPLE

### Example 1

### Antiviral Agent and Hair Growth

A 48 year old bald male subject (Norwood Level VII) who routinely gets fever blisters volunteered to take place in a study to see if the topical application and oral consumption of antiviral drugs would result in hair growth. The individual lacks any hairs on the top of his head. The participant was instructed to cleanse his scalp twice daily with a liquid cleanser using a sonic skincare brush (Clarisonic®). After cleansing topical acyclovir (5%) was applied. The subject is not a candidate for any of the FDA-approved hair loss medications because of a lack of vellus hairs.

After two weeks of treatment, the participant noted small vellus hairs and tactically noticed new hair sensation. A marked increase in oral lesions also occurred, and the participant was give oral acyclovir to suppress viral replication. After approximately four weeks of treatment, the participant was given valacyclovir (500 mg) instead of oral acyclovir because of potential acyclovir resistance. In addition, the concentration of topical acyclovir was increased to 10%.

At 12 weeks, long, unpigmented vellus hairs were visible on the scalp. After 16 weeks of treatment, some of the vellus hairs converted to pigmented hairs. After 20 weeks, the pigmented and unpigmented vellus hairs continued to lengthen. The fact that vellus hair formation occurred and converted to pigmented hairs is highly unexpected in a participant with Norwood Level VII baldness.

After 24 weeks of treatment, the subject switched to an acyclovir-glycol:alcohol base formulation for two weeks, and stopped oral antiviral medication for five days. The topical acyclovir precipitated and did not appear to penetrate the scalp. During this time frame the vellus hairs were shed lending support to a viral component to hair loss.

While illustrative embodiments have been illustrated and described, it will be appreciated that various changes can be made therein without departing from the spirit and scope of the invention.

1. A method of predicting the likelihood that a subject will develop alopecia, comprising:
(a) determining a presence of a nerve dwelling virus in a subject;
(b) determining a level of at least one of 5α-dihydrotestosterone, 5α-reductase, or testosterone in the subject;
(c) comparing the level of 5α-dihydrotestosterone, 5α-reductase, or testosterone determined from step (b) to a predetermined value; and
(d) predicting alopecia or a predisposition to alopecia in the subject, wherein an elevated level of 5α-dihydrotestosterone, 5α-reductase, or testosterone in comparison to the predetermined value and the presence of the nerve dwelling virus is indicative of an increased likelihood of the subject developing alopecia.

2. The method of claim 1, wherein the nerve dwelling virus is herpes simplex virus.

3. The method of claim 1, wherein the presence of the nerve dwelling virus in the subject is determined by an assay selected from
(a) an immunological assay;
(b) a polymerase chain reaction assay; or
(c) an RNA-DNA microarray assay.

4. The method of claim 1, wherein the level of 5α-dihydrotestosterone, 5α-reductase, or testosterone in the subject is determined by an assay selected from
(a) an immunological assay;
(b) a polymerase chain reaction assay; or
(c) an RNA-DNA microarray assay.

5. A method for identifying an individual as a candidate for treatment of androgenetic alopecia, comprising:
(a) obtaining a positive test result for a herpes simplex virus in an individual;
(b) determining the level of at least one of 5α-dihydrotestosterone or 5α-reductase;
(c) comparing the level of 5α-dihydrotestosterone or 5α-reductase to a predetermined value; and
(d) characterizing the individual as a candidate for treatment of androgenetic alopecia based upon a positive test result in step (a) and an elevated level of 5α-dihydrotestosterone or 5α-reductase in comparison to the predetermined value.

6. The method of claim 5, wherein the herpes simplex virus is HSV-1.

7. A method for identifying a male subject infected with herpes simplex virus as a candidate for treatment of androgenetic alopecia, comprising:
(a) measuring a level of at least one of 5α-dihydrotestosterone or 5α-reductase in a male subject infected with herpes simplex virus;
(b) comparing the level measured in step (a) to a reference, wherein a level above the reference indicates that the male subject is a candidate for treatment of androgenetic alopecia.

8. A method of managing a treatment of androgenetic alopecia in a subject, comprising the steps of:
(a) measuring the presence of active herpes simplex virus replication in a subject;
(b) measuring a level of at least one of 5α-dihydrotestosterone or 5α-reductase in the subject;
(c) comparing the level from step (b) to a predetermined value; and
(d) adjusting the treatment of the subject to suppress replication of the herpes simplex virus or to reduce the level of at least one of 5α-dihydrotestosterone or 5α-reductase.

9. The method of claim 8, wherein the herpes simplex virus is HSV-1.

10. A kit for predicting or monitoring androgenetic alopecia in a subject, comprising:
(a) at least one reagent for detecting herpes simplex virus in a biological sample;
(b) at least one reagent for detecting or measuring the level of 5α-dihydrotestosterone or 5α-reductase in a biological sample;
(c) a reference standard for the assay of step (b);
(d) a set of instructions describing how to assay the biological samples; and
(e) a chart for determining the likelihood of the subject developing androgenetic alopecia based upon the test results obtained from the biological samples.

11. The kit of claim 10, wherein the herpes simplex virus is HSV-1.

12. The kit of claim 10, wherein the kit comprises at least one collection device.
